(19) United States
(12) Patent Application Publication
(10) Pub. No.: US 2024/0023815 A1
(43) Pub. Date: Jan. 25, 2024

(54) PORTABLE, MULTI-FUNCTIONAL HEALTH MONITORING DEVICE

(71) Applicant: QINGDAO YASEE MEDICAL EQUIPMENT CO., LTD., Qingdao (CN)

(72) Inventors: Zhaowang HAN, Qingdao (CN); Xinming CHEN, Qingdao (CN)

(21) Appl. No.: 18/255,744

(22) PCT Filed: Dec. 3, 2021
(86) PCT No.: PCT/CN2021/135547

§ 371 Date: Jun. 2, 2023

(30) Foreign Application Priority Data
Dec. 4, 2020 (CN) 202011416632.8
Dec. 8, 2020 (CN) 202011425601.9
Feb. 10, 2021 (CN) 202110183775.7
Feb. 10, 2021 (CN) 202110186436.4
Feb. 10, 2021 (CN) 202110186437.9

(51) Int. Cl. A61B 5/0205

(52) U.S. Cl. CPC A61B 5/0205; A61B 2560/0431; A61B 2560/0443; A61B 2560/0223; A61B 2562/08; A61B 5/1477

(57) ABSTRACT

A portable, multi-function health detector, primarily comprises: a primary block (100), being configured to output and/or store detection data associated with various detection tasks, a primary interface (101), being installed on the primary block (100) and configured to establish adaptive connection with distinct, individual split detection blocks (200), and at least one split detection block (200), being connected to the primary block (100) through a split interface (201) that is adapted to the primary interface (101), so as to perform a specific kind of detection tasks, wherein, the primary block (100) is externally connected to at least one split detection block (200) through the primary interface (101) while having at least one built-in detection unit (104), in which, only when connected to the primary block (100), is the split detection block (200) enabled to perform the specific kind of detection tasks, and the specific kind of detection tasks performed by the split detection block (200) is different from the detection tasks of the detection unit (104) of the primary block (100).

### BACKGROUND OF THE INVENTION

### 1. Technical Field

This invention relates to devices for health monitoring, and more particularly to a portable, multi-functional health monitoring device.

### 2. Description of Related Art

CN202776279U discloses a multi-functional health monitoring device, which processes multiple detection items simultaneously and displays a large number of measurements in a single display screen (as shown in FIG. 4 of the referred patent document). This existing monitor features for its scalability and this limits its portability to the bare core structure. It is clear that once the peripheral detecting devices for electrocardiography, pulse, heart rate, body temperature, blood pressure, blood glucose, blood fat and more are connected to the core structure, the overall system unavoidably becomes too bulky to be portable. Moreover, according to the specification and drawings provided in the referred patent document, the prior-art equipment is actually a terminal for collecting physiological indicators from various existing detecting devices, processing the data, and displaying the data. This earlier disclosure nevertheless provides no reasonable setting that is required by its connection to and use with external detecting devices. For example, it fails to explain how its MCU and A/D conversion module can be reasonably linked to the detector and external detecting devices for optimal detection. Also, it fails to address data connection and data processing to minimize the number of involved electronic components. Power supply to external detecting devices is another problem left unsolved.

CN211122887 discloses an analyzer with split structure formed by a host connected with separate measurement modules. While the earlier disclosure asserts advantages of easy component replacement and reduced maintenance costs, it provides no details about data connection of its measurement modules, of its host, and between the host and the measurement module from the perspective of system design. For example, as the host of the system is functionally a terminal for outputting, processing, and storing data, the host itself possesses no detection capability unless it is connected to at least one measurement module. Another potential problem is that to make different types measurement modules link to the host and form a network, the host and these measurement modules have to be adapted in terms of mechanical and electronic structure and connection among them, which leads to increased design works and manufacturing costs. Additionally, since the measurement modules having different functions may be of diverse forms, they have to be equipped with dedicated power suppliers and display screens, respectively, which unavoidably increases the size, cost, and power consumption and advise to the portability of the overall system.

There have been many split detection systems developed for healthcare applications, and most of them use separate detection devices separated from a host that are functionally and operationally standalone. In this case, a host only works as a hub for collecting data from the detection device in a wireless or wired manner. As the host and the detection devices are separate and each standalone, there are many equipment overlaps in terms of, for example, power source, data storage, operation, and display. Such overlaps not only mean increased hardware/software costs, but also objectively degrade the portability of the overall system. For an application where the host is to be used with a dozen or so detection devices, the resulting system may be rejected from boarding an airplane whether as a check-in luggage or carry-on luggage for it contains too many lithium batteries.

Hence, there is a need for a portable detector that has split detection blocks that can be stored separately, or stated differently, is a split-type detector. The split detection blocks work with a primary block to form a really portable and multi-function system for interactive health detection.

### SUMMARY OF THE INVENTION

For achieving the above objective, the present invention provides a portable, multi-function health detector, which primarily comprises: a primary block, being configured to output and/or store detection data associated with various detection tasks; a primary interface, being installed on the primary block and configured to establish adaptive connection with distinct, individual split detection blocks; at least one kind of the split detection blocks, each being connected to the primary block through a split interface that is adapted to the primary interface so as to perform a specific kind of detection tasks.

Preferably, the primary block is externally connected to at least one said split detection block through the primary interface while having at least one built-in detection unit (104). Therein, only when connected to the primary block, can the split detection block perform corresponding said detection tasks, and the detection tasks performed by the split detection block perform are different from those of the detection unit of the primary block. The primary block and the split detection block have different types of detection units provided for detection tasks to measure different physiological indicators. This means that the two jointly form a versatile detector system, wherein every split detection block is in adaptive connection with the primary block. Thus, the resulting detector is extensively applicable, making it a single system for multiple purposes.

Preferably, the present invention provides a portable, multi-function health detector, which primarily comprises a primary block having an electrochemical detection unit and/or an oscillometric detection unit for outputting at least one kind of health data; and at least one split detection block for outputting at least one further kind of health data that is different from the health data output by the electrochemical detection unit and/or the oscillometric detection unit. Therein, only when electrically connected to the outside of the primary block and driven by the primary block, is the split detection block enabled to generate the at least one further kind of health data. The primary block primarily comprises an electrochemical detection unit for measuring blood glucose, blood ketone and uric acid and/or an oscillometric detection unit for measuring blood pressure, and the electrochemical detection unit and/or the oscillometric detection unit is connected to the operational analysis unit in the primary block. Many physiological indicators such as blood glucose, blood ketone, uric acid, and blood pressure are common concerns of people as these measurements are very sensitive to health states of individuals. With measurement of these common physiological indicator incorporated therein as its basic functions, the primary block alone can satisfy healthcare needs of most people. Also, the primary block is structurally and operationally uncomplicated, making it friendly to various kinds of users. Additionally, the primary block can work with various split detection blocks to form a multi-function detector suitable for various applications where different physiological indicators have to be measured. A user can personalize his/her own detector by incorporating different detection blocks supporting different detection tasks. Meanwhile, since all the individual split detection blocks are operated and displayed using the common user interface on the primary block, they can be free from desiccated yet repeated operational/display components, and this significantly reduces design works and manufacturing costs, while the resulting terminal devices are allowed to be made compact and lightweight. Moreover, the electrochemical detection unit and/or the oscillometric detection unit is most preferably installed on the primary block because detection performed by the two components requires seif-calibration, which is relatively time-consuming, whereas the other split detection blocks generally function as optical or physical measurement units, which are more suitable for plug-and-play use.

Preferably, the primary block and/or the split detection block is provided with an operational analysis unit that is configured to process the detection data from the detection unit and/or detection data associated with the detection tasks of at least one said split detection block that is in connection with the primary block.

Preferably, the operational analysis unit is connected to and/or integrated with at least one analog-to-digital converter that is configured to receive the detection tasks data from the primary block and/or from the split detection block through a sub-channel manner, and the analog-to-digital converter converts and transmits the detection data to the operational analysis unit for analysis and processing.

Preferably, the primary block is provided with a port for detection and/or calibration, such that upon insertion of a specific test paper and/or a standard test paper into the port, the specific detection data and/or standard data is recorded by the health detector, so as to at least obtain a value of a relevant physiological indicator measured according to an electrochemical detection principle.

Preferably, the at least one kind of said split detection block contained in the health detector is configured to be connected to the primary block in a one-on-one manner according to a specific detection need, in which, the split detection block primarily comprises a block selected from the following: a blood oxygen detection block, a blood glucose detection block, a blood pressure detection block, a blood fat detection block, a dry chemistry detection block, a pulse, fluorescence and colloidal gold method detection block, an electrocardiography detection block, a body fat detection block, a fetal heart detection block, a breast milk analysis detection block, a gas content detection block, and a pulmonary function and peak flow detection block

Preferably, the split detection block is provided with an ID module, such that the split detection block in connection with the primary block is enabled to provide module ID data associated with the ID module to the primary block in response to said connection, in which the module ID data enables identification of the split detection block.

Preferably, the health detector has a working mode determined according to the module ID data by the primary block, and the determined working mode is activated through manual selection made by a user and/or through automatic adaption made by the primary block.

Preferably, the primary block can determine a data processing mode for the operational analysis unit based on the working mode determined according to the module ID data, and particularly the data processing mode is for working with data from the split detection block adaptively connected to the primary interface. Each of the primary block and the split detection blocks is capable of measuring a certain number of physiological indicators. The primary block determines the current working mode of the detector according to the split detection block connected thereto, and controls the corresponding parts of the detector, thereby preventing repeated operations and improving detection efficiency.

Preferably, only when connected to the primary block, can the split detection block perform detection tasks in response to the corresponding operation at the primary block. The split detection block and the primary block work together to perform the detection tasks. The split detection block will not perform any detection without the conformation from the primary block. This helps prevent potential waste of resources and thereby reduce power consumption. Specifically, since the battery and the switch on the primary block are limited in service life, the foregoing on-demand/plug-in activation is at least advantageous from the perspective of minimization of both power consumption and maintenance needs. Besides, in the prior art, while the split-type structure is often contributive to functional flexibility, it exponentially increases complexity of the resulting system in terms of hardware and operation. On the contrary, in the present invention, the split detection blocks share a single primary block (meaning that only the primary block has the sole display portion and a minimal number of operational components, and the split sides need no these components), such that the time required by learning the detector before use can be significantly reduced, making the system even more friendly to various kind of users. At last, with the most common detection functions built therein, the primary block alone can satisfy daily healthcare needs for most users. For users who have individual additional measurement needs, the detector can be personalized by incorporating detection blocks with corresponding functions. As the number of functions is limited to exactly what the user needs, this eliminates redundant functions that adds unnecessary costs and bulkiness.

Preferably, the split interfaces installed on the split detection blocks that are adaptively connected to the primary block to perform different kinds of said detection tasks are identical to each other.

Preferably, the primary block is provided with a power source, such that only when connected to the primary block through the split interface, is the primary block enabled to power the split detection block. In other words, the split detection block has not to be equipped with a battery or charging components. Instead, it only draws power form a connected device when starting to perform detection. This arrangement contributes to decreased equipment size and increased portability to some degree. By eliminating iteration of power supplies, the present invention reduces manufacturing costs and power consumption. It is of great help particularly when the detector is carried by a user who travels with airplanes a lot because the disclosed detector only uses one power source and is travel safe, opposite to conventional ones that contain too many lithium batteries to be allowed to board airplanes. Consequently, the disclosed detector is really portable as it can go anywhere with its users. Another point is that there is only one device, namely the primary block, needing charging, so the “out-of-battery” risk during operation is minimized. Even if the detector needs to be charged or has to work while plugged in, it takes up only one charging line for the primary block only. This is particularly advantageous in terms of electrical safety and resource conservation. Furthermore, since a battery is the part requires replacement most frequently in an electronic product, removal of power supplies from the split detection blocks means significant saving in maintenance and great reduction in environmental impact. At last, in the prior art, the primary block has to either be equipped with a large number of detection units or work with numerous standalone detection devices such as plug-in medical monitors, and these attachments all necessitate powerful processing capability and sophisticated user interface from the primary block, making the costs exponentially increased. On the contrary, in the present invention, as activation only happens when connection has been established, the primary block only has to take care of a few kinds of detection tasks. Therefore, primary block can be realized using a simple, inexpensive CPU and a limited storage and simple user interface are quite sufficient for its operation.

Preferably, only when connected to the primary block, is the secondary detection block enabled to at least access data in the primary block and/or display information associated with the detection tasks through a display portion of the primary block in response to an operation of the primary block instructing so.

Preferably, the primary block is provided with a wireless communication module that is configured to transmit/receive data associated with the split detection block and/or the detection unit and/or an external instruction to/from an external device. The split detection blocks do not have to be equipped with dedicated display and/or operational components, as they are configured to function through the related functional portions provided on the primary block. This minimizes overlaps between the split detection blocks and the primary block in terms of hardware and function, thereby objectively reducing costs while endowing the overall system with maximized portability.

Preferably, the primary block and/or the split detection blocks may be provided with at least one data collector that is configured to collect data and transmit the collected data in an unprocessed form to the analog-to-digital converter where the data is converted and then provided available data to the operational analysis unit.

### BRIEF DESCRIPTION OF THE DRAWINGS

FIG. 1 is a block diagram of a portable, multi-function health detector showing a preferred connection scheme thereof according to one embodiment of the present invention;

FIG. 2 is a block diagram of a split detection block of the health detector showing a preferred structure thereof; and

FIG. 3 is a block diagram of a primary block of the health detector showing a preferred structure thereof.

### LIST OF REFERENCES

100: primary block; 101: primary interface; 102: operational analysis unit; 103: port; 104: detection unit; 104-a: electrochemical detection unit; 104-b: oscillometric detection unit; 106: analog-to-digital converter; 107: power source; 108: storage portion; 109: display portion; 110: operation portion; 111: wireless communication module; 200: split detection block; 201: split interface; 202: data collector; 203: ID module.

### DETAILED DESCRIPTION OF THE INVENTION

The present invention will be described in detail with reference to the accompanying drawings.

As used herein, a split detection block refers to a split detection block that can be stored separately (or a detection block existing as a split part of the system). Such a separate or split part can be connected to or combined with the portable, multi-function health detection system to perform corresponding detection tasks.

As used herein, a primary block refers to a main body module for connecting with the aforementioned at least one detection block. The primary block is equipped with a power unit (a battery or a power source), an analysis unit (MCU), a storage unit (EEPROM), a feedback unit (a visual, auditory and/or tactile sense) and/or an operation unit for transmitting or receiving data, instruction and/or signaling to or from the connected detection block, for authenticating the connected detection block, for powering the connected detection block, for restarting/resetting the connected detection block, for testing or marking the connected detection block, and so on.

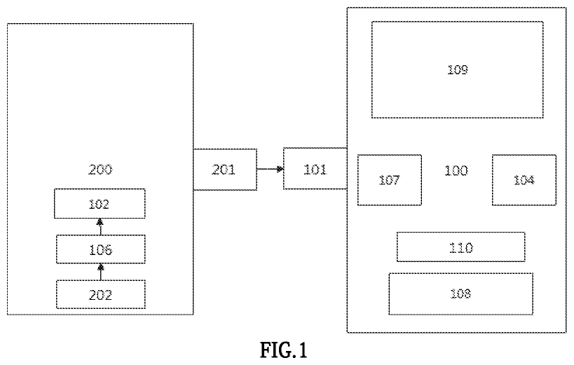

According to one preferred implementation, the present invention provides a portable, multi-function health detector. The health detector may comprise one of the following components: a primary block 100, a standardized primary interface 101 installed on the primary block 100, a split detection block 200, and a split interface 201 installed on the split detection block 200, as shown in FIG. 1.

According to one preferred implementation, the primary block 100 is integrated therein at least one detection unit 104, so that the primary block 100 performs detection tasks for measuring at least one physiological indicator through the detection unit 104. The primary block 100 has externally at least one primary interface 101, so that the primary block 100 can be adapted to different split detection blocks 200 through the primary interface 101 at least in terms of current signal and/or mechanical combination.

According to one preferred implementation, the split detection block 200 is different from the detection unit 104 in the primary block 100. In other words, the split detection block 200 and the primary block 100 are designed for different detection tasks. Specifically, the split detection block 200 is connected to the primary block 100 through the split interface 201 adapted to the primary interface 101 so as to realize mechanical connection and/or signal transmission between itself and the primary block 100, thereby being able to perform the detection tasks different from those of the detection unit 104 in the primary block 100. Only when connected to the primary block 100, can the split detection block 200 perform corresponding detection.

According to one preferred implementation, the detection unit 104 in the primary block 100 is provided with at least one circuit board, on which at least an analog-to-digital converter 106 and an operational analysis unit 102 (i.e., a central processing unit, or CPU) are installed. Specifically, the analog-to-digital converter 106 may be integrated in the operational analysis unit 102 on the circuit board or may be independently installed on the circuit board, so as to convert raw, unconverted/unprocessed data into a data type that the operational analysis unit 102 can process. The operational analysis unit 102 can process the detection data that comes from the at least one kind of detection unit 104 integrated in the primary block 100 and has been converted by the analog-to-digital converter 106 and/or detection data that comes from at least one split detection block 200 connected to the primary block 100. The operational analysis unit 102 may be a digital signal processor (DSP), a micro control unit (MCU) or a micro-processor unit (MPU). The operational analysis unit 102 may be integrated with on-chip peripheral components or connected externally to peripheral components (such as a memory array).

Preferably, the primary block 100 and/or the split detection block 200 is provided therein with at least one data collector 202 that collects data and send the collected data in an unprocessed form to the analog-to-digital converter 106 for conversion and then to the operational analysis unit 102 for analysis and processing. Furthermore, the split detection blocks 200 perform distinct detection tasks depending on the kinds of the data collector 202 integrated with them. The data collector 202 may be a sensor or another detection unit that is well known to people skilled in the art and can perform data collection.

According to one preferred implementation, the at least one detection unit 104 integrated into the primary block 100 primarily comprises an electrochemical detection unit 104-a for measuring physiological indicators such as blood glucose, blood ketone and/or uric acid, and/or an oscillometric detection unit 104-b for measuring blood pressure. In addition, the primary block 100 is provided externally with at least one port 103 for detection and/or calibration, so that after a specific test paper and/or standard test paper is inserted into the port 103, the related detection data and/or standard data can be recorded into the detector, so as to obtain a value of a physiological indicator measured according to an electrochemical detection principle. Besides, in actual detection, a user can compare the measured data and/or curves with a corresponding standard value with the help of the port 103, thereby enhancing accuracy of detection.

According to one preferred implementation, for measuring physiological indicators that can be measured electrochemically, such as blood glucose, blood ketone and/or uric acid, the operation involves, for example, applying the blood specimen of a subject to be tested (e.g., a patient) to a specified area in a specific test paper, and then inserting the specific test paper carrying the blood specimen into the port 103 of the primary block 100. The data collector 202 in the primary block 100 can collect information of the blood specimen of the subject to be tested from the specific test paper, and send the information to the analog-to-digital converter 106 for data conversion. The converted data is afterward collected for processing and analysis at the operational analysis unit 102, so as to generate measurement and exam results about the physiological indicators (i.e., blood glucose, blood ketone and/or uric acid) of the subject to be tested.

According to one preferred implementation, for measuring physiological indicators that can be measured oscillometrically, such as blood pressure, the primary block 100 may be provided with a blood-pressure cuff port that is connected to the data collector 202. Specifically, the operation involves putting the blood pressure cuff around the arm of the subject to be tested, so that the numeral value reflecting the variation of the blood pressure can be collected by data collector 202 (e.g., a pressure sensor in this case) installed in the blood-pressure cuff and sent to the analog-to-digital converter 106 for data conversion. The converted data is then collected and analyzed by the operational analysis unit 102, so as to generate measurement and exam results about the physiological indicator(i.e., blood pressure) of the subject to be tested.

According to one preferred implementation, the primary block 100 may further include a power source 107, a storage portion 108, a display portion 109 and an operation portion 110 for the primary block 100 and/or the split detection block 200 to use, as shown in FIG. 1.

Preferably, the power source 107 in the primary block 100 is configured to at least power the uncharged split detection block 200 adaptively connected thereto through the primary interface 101. In other words, the split detection block 200 does not have to be equipped with a dedicated battery and thus has no charging needs. The split detection block 200 only draws power from an external device, such as the primary block 100 of the present invention, when it starts to perform a detection task.

According to one preferred implementation, detection tasks data from the primary block 100 and/or the split detection block 200 that has been processed by the operational analysis unit 102 can at least be output through the display portion 109 provided on the primary block 100. Alternatively, the processed data may be output through a third-party display that is communicatively coupled with the primary block 100.

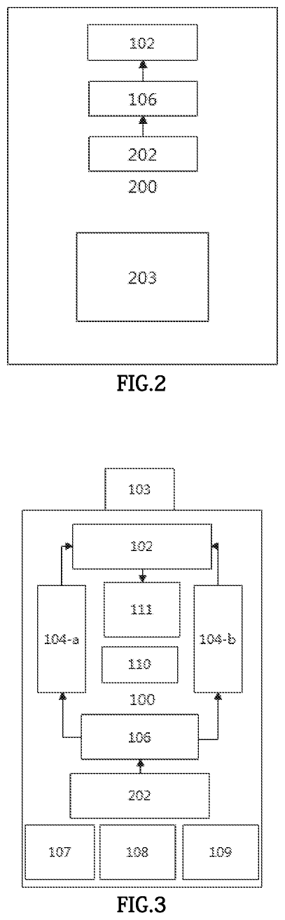

According to one preferred implementation, the split detection block 200 works with the primary block 100 to form the disclosed portable, multi-function health detector for desired detection. The split detection block 200 may comprise one of the following: split interface 201, data collector 202, analog-to-digital converter 106, operational analysis unit 102, and an ID module 203, as shown in FIG. 2.

According to one preferred implementation, the split detection block 200 may be internally provided with an ID module 203. The ID module 203 is preloaded with multiple sets of module ID data corresponding to different types of data collectors 202, such that when a corresponding split detection block 200 is connected to the primary block 100, the split detection block 200 at least can provide the primary block 100 with the module ID data associated with the ID module 203 that can be used to identifying the identity information specific to the split detection block 200 in response to the foregoing docking. According to the module ID data, the working mode of the detector can be determined through manual selection and/or automatically by the primary block 100 automatic.

According to one preferred implementation, the split interface 201 of the split detection block 200 conforms to the current USB specification, and may be a mini-USB port, a micro-USB port or a Type-C port. The USB specifications may include but is not limited to USB 1.0, USB 1.1, USB 2.0, USB 3.0 (USB 3.1 Gen1/USB 3.2 Gen1), USB 3.1 (USB 3.1 Gen2/USB 3.2 Gen2×1), USB 3.2 (USB 3.2 Gen2×2), USB 3.2 Gen 1×2, USB 3.2 Gen 2×2, USB4, USB On-The-Go Supplement Revision and USB Power Delivery. Also included is Wireless Universal Serial Bus (Wireless USB). In addition, for convenient use, the split interface 201 may preferably be a Type-C interface, so as to dock the split detection block 200 to the primary block 100 simply and rapidly.

Preferably, when the split detection block 200 is connected to the primary block 100 through the split interface 201, depending on the types of the split detection blocks 200, they have particular types of identification information for identifying their identities, so the primary block has to be set the appropriate working mode for itself to dock to the relevant split detection block. In an example where the split detection block 200 needs to send an identity authentication request to the primary block 100 through the split interface 201, the identity authentication request is born by the module ID data (i.e., any data that is suitable for indicating the ID, such as a manufacturer code, a device model, hardware codes of its components, and a function serial number). Further, when the primary block 100 receives an identity authentication request from the split detection block 200, i.e., the module ID data, the primary block 100 compares the module ID data with the standard identity information that has been stored in the primary block 100 in advance, and determines the split detection block 200 is of which type according to how the module ID data and the standard identity information match each other, thereby determining the exact working mode of the detector.

Preferably, the primary block 100 determines the particular type of the split detection block 200 adaptively connected through the primary interface 101 using the ID module 203 in the split detection block 200, thereby determining the current working mode of the detector. When the specific kind of detection tasks performed by the split detection block 200 adaptively connected to the primary block 100 is different from the detection function of the detection unit 104 in the primary block 100, the primary block 100 can, after determining he particular structure and functional type of the split detection block 200 based on the module ID data, automatically select the current working mode of the detector. Alternatively, the current working mode of the detector can be selected manually so that the subsequent detection task and data processing as well as analysis can be done.

According to one preferred implementation, the split detection block 200 at least can, when connected to the primary block 100 through the split interface 201, send the corresponding detection data to the primary block 100 for output and/or storage. Regardless their different types, all the split detection blocks 200 use identical split interfaces 201. The primary block 100 can determine the data processing mode for the operational analysis unit 102 in response to the determination of its own working mode, particularly for the processing mode for data coming from the split detection block 200 in connection with the primary interface 101.

According to one preferred implementation, the primary block 100 may be provided therein with a wireless communication module 111. The wireless communication module 111 is used to receive and transmit data and/or external instructions associated with the split detection block 200 and the detection unit from and to an external device.

Furthermore, since the split detection block 200 is designed to provide a single specific function, its size can be minimized. Consequently, when the split detection block 200 is connected to an external device, such as the primary block 100 as disclosed herein, the combination of the two remains relatively compact and thus portable. The primary block 100 uses the sole primary interface 101 to connect different single-function split detection block 200, so the resulting system as a whole is highly economical and portable.

Preferably, if the split detection block 200 is a blood oxygen split detection block adaptively connected to the primary block 100 for detecting the oxygen content in the blood of a subject to be tested, the blood-oxygen-specific split detection block is equipped with a data collector 202 such as a sensor or something alike. The split detection block 200 uses at least two light-emitting diodes built in the sensor to emit light beams of different wavelengths to a tested site of the subject to be tested. According to variations in light absorption at different wavelengths caused by the binding of oxygen to globins, the proportion of globins in the blood can be measured, and then the detection data can be sent to the operational analysis unit 102, thereby determining the oxygen level in the blood of the subject to be tested.

Preferably, if the split detection block 200 is a blood-glucose-specific split detection block that is adaptively connected to the primary block 100 for detecting the level of glucose in the blood of a subject blood, the split detection block specific to blood glucose is provided with a data collector 202 such as a sensor. The split detection block 200 uses at least three electrodes built in the sensor, namely a working electrode, a reference electrode, and a comparison electrode, to measure the electric current generated as a result of the chemical reaction between glucose in the blood of the subject to be tested and glucose oxidase contained in the test paper, and then send the detection data to the operational analysis unit 102, thereby determining the blood glucose level of the subject to be tested.

Preferably, if the split detection block 200 is a blood-pressure-specific split detection block that is adaptively connected to the primary block 100 for measuring the blood pressure of a subject to be tested, the blood-pressure-specific split detection block has a built-in data collector 202 such as a sensor or something alike. The split detection block 200 uses the built-in pressure sensor to measure the systolic pressure and/or diastolic pressure of the subject to be tested when the blood passes through the cuff, and then sends the detection data to the operational analysis unit 102 for determining the blood pressure of the tested subject.

Preferably, if the split detection block 200 is an electrocardiography-specific split detection block that is adaptively connected to the primary block 100 for detecting the heart rate of a subject to be tested. The electrocardiography-specific split detection block has a built-in data collector 202 such as a sensor or something alike. The split detection block 200 uses electrodes built in the sensor to measure the variations in electric potential difference between two sites at the surface of the human body, and then sends the detection data to the operational analysis unit 102 for determining the heart rate of the subject to be tested and in turn ascertaining the working state of the heart of the subject to be tested.

Preferably, if the split detection block 200 is a blood-cholesterol-specific split detection block that is adaptively connected to the primary block 100 for measuring indicators of lipoids in the blood of the subject to be tested, the split detection block has a built-in data collector 202 such as a sensor or something alike. The split detection block 200 uses the detection plated built in the sensor and/or the lancet and the test strip coming with the blood fat tester to get measurements generated from the reactions between some certain substances in the blood (such as cholesterol and triglyceride) and a reactant, and then sends the detection data to the operational analysis unit 102, thereby determining he levels of lipids in the blood of the subject to be tested.

Preferably, if the split detection block 200 is a progesterone-specific split detection block that is adaptively connected to the primary block 100 for detecting the level of progesterone in the blood of a subject to be tested, the split detection block has a built-in data collector 202 such as a sensor or something alike. The split detection block 200 uses the fluorescence probe solution in the sensor to measure the intensity of fluorescence, and sends the detection data to the operational analysis unit 102, thereby determining the level of progesterone in the blood of the subject to be tested.

Preferably, if the split detection block 200 is a gas-content-specific split detection block that is adaptively connected to the primary block 100 for detecting the air exhaled by a subject to be tested, the gas-content-specific split detection block has a built-in data collector 202 such as a sensor or something alike. The split detection block 200 uses the ion chamber, the UV lamp and the collection electrode built in the sensor to measure the electric current, and sends the detection data to the operational analysis unit 102, thereby determining the gas contents in air exhaled by the subject to be tested.

Preferably, if the split detection block 200 is a body-fat-specific split detection block that is adaptively connected to the primary block 100 for detecting the body fat rate of subject to be tested. The body-fat-specific split detection block has a built-in data collector 202 such as a sensor or something alike. The split detection block 200 passes power to the metal electrode and/or the ITO conductive film built in the sensor to measure the proportions of body fat, proteins and carbohydrates in the body of a subject to be tested. Since fat is non-conductive, the electric current in the closed circuit varies accordingly, so the operational analysis unit 102 can process the measurement and determine the body fat rate of the subject to be tested.

Preferably, if the split detection block 200 is a breast-milk-specific split detection block that is adaptively connected to the primary block 100 for detecting the content of the breast milk of a subject to be tested, the breast-milk-specific split detection block has a built-in data collector 202 such as a sensor or something alike. The split detection block 200 uses ultrasonic sensing unit and/or infrared sensing unit built in the sensor to identifying the contents of the milk, and determining measurements based on the feedback ultrasonic signal. Then the operational analysis unit 102 processes the association among the levels of the different contents in the milk to determine the contents of the breast milk of the subject to be tested.

Preferably, if the split detection block 200 is a pulmonary-function-specific split detection block that is adaptively connected to the primary block 100 for assessing the pulmonary function of the subject to be tested, the detection block has a built-in data collector 202 such as a sensor or something alike. The split detection block 200 uses the peak flow unit and/or the pointer built in the sensor to measure the pulmonary function peak flow.

For better understanding of the portable multi-function health detector, the working principle and use of the multi-function health detector of the present invention will be detailed below.

In use of the detector disclosed herein, for measuring physiological indicators of a subject to be tested such as blood glucose and blood ketone that can be measured electrochemically and/or physiological indicators such as blood pressure that can be measured oscillometrically, the port 103 installed on the primary block 100 and the detection unit 104 integrated in the primary block 100 can be used to do the measurements, and the measures can be at least output through the display portion 109. On the other hand, when a physiological indicator that is not one can be measured by the detection unit 104 of the primary block 100 is to be measured, the split interface 201 of a split detection block 200 specific to that indicator can be removably attached to the primary interface 101 of the detector, so as to get power and/or data communication. The split detection block 200 can send the module ID data associated with the ID module 203 therein to the primary block 100 through the split interface 201, and then the primary block 100 can determine the particular type of the currently connected split detection block 200 according to the module ID data coming from the split detection block 200, thereby determining the working mode appropriate to the current application. The split detection block 200 can perform a detection task and/or a resetting task prior to the intended detection task in response to its connection with the primary block 100, and send data associated with the detection task and/or the resetting task to the operational analysis unit 102, so that measurement of a physiological indicator that is not supported by the primary block itself can be determined and/or calibrated. The split detection block 200 can then send the final detection result to the primary block 100 through the split interface 201 and make the result output at least through the display portion 109.

The health detector of the present invention is inexpensive, compact, and lightweight, making it highly portable to be used in various occasions. The detection blocks of different functions can be easily incorporated in the system and replaced according to practical needs to measure an extensive range of physiological indicators for better healthcare.

1-15. (canceled)

16. A portable, multi-function health detector, at least comprising:
a primary block, being configured to output and/or store detection data associated with various detection tasks,
a primary interface, being installed on the primary block and configured to establish adaptive connection with distinct, individual split detection blocks, and
at least one said split detection block, being connected to the primary block through a split interface that is adapted to the primary interface, so as to perform corresponding detection tasks,
wherein,
the primary block is externally connected to at least one said split detection block through the primary interface while integrated therein at least one detection unit,
in which,
only when connected to the primary block, is the split detection block enabled to perform the corresponding detection tasks, and the detection tasks performed by the split detection block is different from the detection tasks of the detection unit of the primary block, the split detection block and the primary block work together to perform the detection tasks, the split detection block which is the on-demand/plug-in activation will not perform any detection without the conformation from the primary block,
the primary block is provided with a power source, such that when the uncharged split detection block is connected to the primary block through the split interface, the primary block is enabled to power the split detection block,
only when connected to the primary block, is the split detection block enabled to at least access and/or store data in the primary block, and/or display information associated with the detection tasks through a display portion of the primary block in response to an operation of the primary block instructing so.

17. The health detector of claim 16, wherein the primary block and/or the split detection block is provided with an operational analysis unit that is configured to process the detection data from the detection unit and/or detection data of at least one said split detection block that is in connection with the primary block.

18. The health detector of claim 17, wherein the operational analysis unit is connected to and/or integrated with at least one analog-to-digital converter that is configured to receive the detection task data from the primary block and/or from the split detection block by way of a sub-channel, and the analog-to-digital converter converts and transmits the detection task data to the operational analysis unit for analysis and processing.

19. The health detector of claim 16, wherein the primary block is provided with a port for detection and/or calibration, such that upon insertion of a test paper and/or a standard test paper into the port, the corresponding detection data and/or standard data is recorded by the health detector, so as to at least obtain a relevant physiological index value measured according to an electrochemical detection principle.

20. The health detector of claim 16, wherein the at least one kind of said split detection block contained in the health detector is configured to be connected to the primary block in a one-on-one manner according to detection need, in which,
the split detection block at least comprises a block selected from the following: a blood oxygen detection block, a blood glucose detection block, a blood pressure detection block, a blood fat detection block, a dry chemistry detection block, a pulse, fluorescence and colloidal gold method detection block, an electrocardiography detection block, a body fat detection block, a fetal heart detection block, a breast milk analysis detection block, a gas content detection block, and a pulmonary function and peak flow detection block.

21. The health detector of claim 16, wherein the split detection block is provided with an ID module, such that the split detection block in connection with the primary block is enabled to provide module ID data associated with the ID module to the primary block in response to said connection, in which the module ID data enables identification of the split detection block,
the health detector has a working mode determined by the judgment result of the primary block on the module ID data, and the corresponding working mode is activated through manual selection and/or through automatic adaption made by the primary block,
the primary block is configured to determine a data processing mode for the operational analysis unit based on the working mode determined according to the module ID data, and particularly the data processing mode for data from the corresponding split detection block adapted to connect with the primary interface.

22. The health detector of claim 16, wherein only when connected to the primary block, is the split detection block enabled to activate one said detection task of the split detection block in response to an operation of the primary block instructing so.

23. The health detector of claim 22, wherein the split interfaces installed on the split detection blocks that are used for adaptively connecting to the primary block to perform different kinds of said detection tasks are identical to each other.

24. The health detector of claim 16, wherein the primary block is provided with a wireless communication module that is configured to transmit/receive data associated with the split detection block and/or the detection unit, and/or an external instruction to/from an external device.

25. The health detector of claim 17, wherein the primary block and/or the split detection block is provided with at least one data collector that is configured to collect data and transmit the collected data in an unprocessed form to the analog-to-digital converter where the data is converted so that usable data is provided to the operational analysis unit.

26. A portable, multi-function health detector, at least comprising:
a primary block having an electrochemical detection unit and/or an oscillometric detection unit, and being configured to output at least one kind of health data,
at least one split detection block that is different from the electrochemical detection unit and/or the oscillometric detection unit, and is configured to output at least one further kind of health data,
wherein,
only when electrically connected to the outside of the primary block and driven by the primary block, is the split detection block enabled to generate the at least one further kind of health data, the split detection block and the primary block work together to perform the detection tasks, the split detection block which is the on-demand/plug-in activation will not perform any detection without the conformation from the primary block,
the primary block is provided with a power source, such that when the uncharged split detection block is connected to the primary block through the split interface, the primary block is enabled to power the split detection block,
only when connected to the primary block, is the split detection block enabled to at least access and/or store data in the primary block, and/or display information associated with the detection tasks through a display portion of the primary block in response to an operation of the primary block instructing so.

27. The health detector of claim 26, wherein the primary block and/or the split detection block is provided with an operational analysis unit that is configured to process the detection data from the detection unit and/or detection data of at least one said split detection block that is in connection with the primary block.

28. The health detector of claim 27, wherein the operational analysis unit is connected to and/or integrated with at least one analog-to-digital converter that is configured to receive the detection task data from the primary block and/or from the split detection block by way of a sub-channel, and the analog-to-digital converter converts and transmits the detection task data to the operational analysis unit for analysis and processing.

29. The health detector of claim 26, wherein the primary block is provided with a port for detection and/or calibration, such that upon insertion of a test paper and/or a standard test paper into the port, the corresponding detection data and/or standard data is recorded by the health detector, so as to at least obtain a relevant physiological index value measured according to an electrochemical detection principle.

30. The health detector of claim 26, wherein the at least one kind of said split detection block contained in the health detector is configured to be connected to the primary block in a one-on-one manner according to detection need, in which,
the split detection block at least comprises a block selected from the following: a blood oxygen detection block, a blood glucose detection block, a blood pressure detection block, a blood fat detection block, a dry chemistry detection block, a pulse, fluorescence and colloidal gold method detection block, an electrocardiography detection block, a body fat detection block, a fetal heart detection block, a breast milk analysis detection block, a gas content detection block, and a pulmonary function and peak flow detection block.

31. The health detector of claim 26, wherein the split detection block is provided with an ID module, such that the split detection block in connection with the primary block is enabled to provide module ID data associated with the ID module to the primary block in response to said connection, in which the module ID data enables identification of the split detection block,
the health detector has a working mode determined by the judgment result of the primary block on the module ID data, and the corresponding working mode is activated through manual selection and/or through automatic adaption made by the primary block,
the primary block is configured to determine a data processing mode for the operational analysis unit based on the working mode determined according to the module ID data, and particularly the data processing mode for data from the corresponding split detection block adapted to connect with the primary interface.

32. The health detector of claim 26, wherein only when connected to the primary block, is the split detection block enabled to activate one said detection task of the split detection block in response to an operation of the primary block instructing so,
the split interfaces installed on the split detection blocks that are used for adaptively connecting to the primary block to perform different kinds of said detection tasks are identical to each other.

33. The health detector of claim 26, wherein the primary block is provided with a wireless communication module that is configured to transmit/receive data associated with the split detection block and/or the detection unit, and/or an external instruction to/from an external device.

34. The health detector of claim 27, wherein the primary block and/or the split detection block is provided with at least one data collector that is configured to collect data and transmit the collected data in an unprocessed form to the analog-to-digital converter where the data is converted so that usable data is provided to the operational analysis unit.

35. An application of the health detector according to claim 26, wherein the health detector is configured to at least be used for at least one or more kinds of blood oxygen detection, blood glucose detection, blood pressure detection, blood fat detection, dry chemistry detection, pulse, fluorescence and colloidal gold method detection, electrocardiography detection, body fat detection, fetal heart detection, breast milk analysis detection, gas content detection, or pulmonary function and peak flow detection.

36. An application of the health detector according to claim 16, wherein the health detector is configured to at least be used for at least one or more kinds of blood oxygen detection, blood glucose detection, blood pressure detection, blood fat detection, dry chemistry detection, pulse, fluorescence and colloidal gold method detection, electrocardiography detection, body fat detection, fetal heart detection, breast milk analysis detection, gas content detection, or pulmonary function and peak flow detection.
